(19) United States
(12) Patent Application Publication
(10) Pub. No.: US 2023/0255510 A1
(43) Pub. Date: Aug. 17, 2023

(54) SPIROMETER ATTACHMENT

(71) Applicant: BRUCE B. HILL, MAPLETON, UT (US)

(72) Inventor: BRUCE B. HILL, MAPLETON, UT (US)

(21) Appl. No.: 17/672,863

(22) Filed: Feb. 16, 2022

(51) Int. Cl. A61B 5/091; A61B 5/00

(52) U.S. Cl. CPC A61B 5/091; A61B 5/002; A61B 5/74; A61B 2560/0431; A61B 2560/0462; A61B 2562/225

(57) ABSTRACT

A spirometer attachment includes a housing. A securement on the housing removably secures the housing to a spirometer. A measuring device within the housing is to collect breath data from the spirometer. At least one LED is to emit light away from the housing based on the collected breath data. A circuit board or controller within the housing is to control the light emitted by the LED based on the breath data.

### BACKGROUND

Spirometers are devices that are used to measure an individual's breathing by measuring the volume and air flow of the individual's lungs when breathing. They may be used in hospital settings, for example, for evaluating patient's with conditions such as asthma, cystic fibrosis, pulmonary fibrosis, and other medical conditions.

### BRIEF DESCRIPTION OF THE DRAWINGS

The accompanying drawings illustrate various implementations of the principles described herein and are a part of the specification. The illustrated implementations are merely examples and do not limit the scope of the claims.

FIG. 1 illustrates a front view of a spirometer attachment according to an example of the principles described herein.

FIG. 2 illustrates a perspective view of a spirometer attachment according to an example of the principles described herein.

FIG. 3 illustrates a perspective view of a spirometer attachment according to an example of the principles described herein.

FIG. 4 illustrates a perspective view of a spirometer attachment according to an example of the principles described herein.

FIG. 5 illustrates a front view of a spirometer, according to an example of the principles described herein.

FIG. 6 illustrates a front view of a spirometer attachment attached to a spirometer, according to an example of the principles described herein.

FIG. 7 illustrates a perspective view of a spirometer attachment attached to a spirometer, according to an example of the principles described herein.

FIG. 8 illustrates a flow diagram with a spirometer attachment, according to an example of the principles described herein.

FIG. 9 illustrates a flow diagram with a spirometer attachment, according to an example of the principles described herein.

### DETAILED DESCRIPTION

The following describes a spirometer attachment that removably attaches to a spirometer and that communicates information about the air flow in the spirometer.

Particularly, the spirometer attachment provides visual information to indicate the amount of air that is being inhaled or exhaled at a various flow data rate or volume. The visual information may be used to guide the user to sustain a pre-determined flow data rate for a pre-determined minimum time period or achieve another breathing objective. For example, the goal may be to increase transpulmonary pressure and inspiratory volumes, improve inspiratory muscle performance, or re-establish or simulate the normal pattern of pulmonary hyperinflation. When the procedure is repeated on a regular basis, airway patency may be maintained and lung atelectasis prevented and reversed.

In an example, a spirometer attachment includes a housing and a securement to removably secure the housing to a spirometer. A measuring device within the housing is to collect breath data from the spirometer. At least one LED (light-emitting diode) is to emit light away from the housing based on the collected breath data. A circuit board or controller within the housing controls the light emitted by the LED based on the breath data.

In another example, a spirometer attachment includes a housing and a securement that removably attaches the housing to a spirometer. A measuring device within the housing is to collect breath data from the spirometer. At least one LED is to emit light away from the housing based on the collected breath data. A circuit board or controller within the housing controls the LED based on the breath data. The breath data is used to determine at least one of rate of breath and volume of lung capacity which is then used to determine the type or mode of LED light to be activated.

In another example, a spirometer attachment includes a housing and a securement that removably attaches the housing to a spirometer. A laser range finder is to measure breath within a portion of space within the spirometer. In an example, the portion of space includes an overlapped space of the housing that is also within the spirometer. At least one LED is to emit light away from the housing based on the collected breath data. A circuit board or controller within the housing controls the LED based on breath data as measured by the laser range finder to guide and teach a user to use the spirometer.

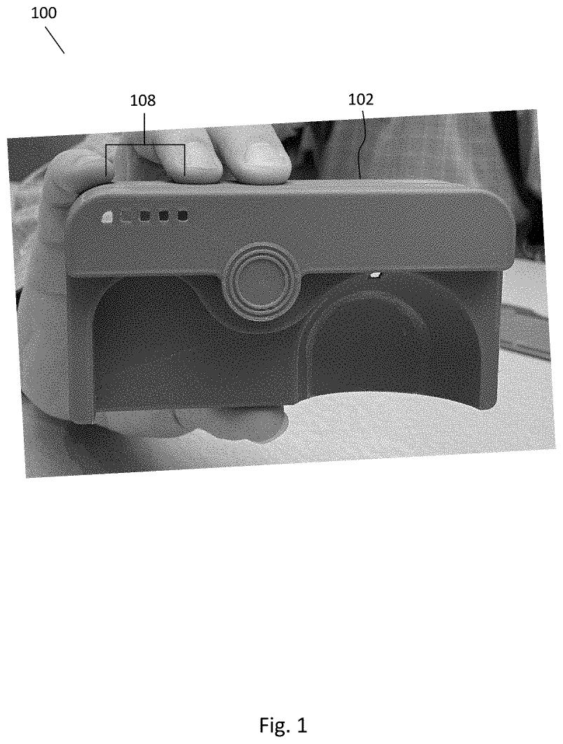

Turning to FIG. 1, a front view of an example spirometer attachment 100 is shown. The spirometer attachment 100 includes a housing 102 that can be attached to a spirometer. The housing 102 includes attachment structure for attaching or otherwise being placed on a spirometer. As shown, the housing 102 includes an interior cavity or space that is within the housing 102. As shown, the back wall and sidewalls of the housing 102 extend downward to fit around the structure of the spirometer. A portion of the front wall also extends partially downward to fit over the front structure of the spirometer. Curvature and contours of the interior surface of the cavity within the housing 102 follow the curvature and contours of the top portion of the spirometer.

In an example, the housing 102 is water-tight or water-resistant such that the spirometer attachment 100 may be removed and sterilized in between uses. This allows hospital rooms and home environments to contain the spread of germs, viruses, bacteria, etc. In another example, the housing 102 is air-tight. The spirometer attachment 100 is thus not a disposable one-time use device.

On the face of the spirometer attachment 100 is a visual indicator to convey visual information related to flow rate or volume based on use of the spirometer. The example visual indicator shown in FIG. 1 is a set of LED lights 108. Each LED light 108 may light up according to an amount of air flow that is being done correctly. In an example, one LED light 108 shows a poor performance while all of the LED lights 108 being lit up indicate a perfect performance. From the initial one LED light 108, each additional LED light 108 lights up as improvement in breathing or other factor is detected. Various types of breathing patterns or exercises may be used with the LED lights 108 lighting up based on performance threshold level or progress.

The shape of the LED light 108 may be a square as shown in FIG. 1. Other shapes may also be used, such as circle, oval, etc. In an example, the LED lights 108 light up a symbolic shape, or form or otherwise define a symbolic shape to users to indicate progress in using the spirometer. Example symbolic shapes include a happy face, sad face, thumbs up, thumbs down, arrow, etc. In an example, a number is presented that indicates the amount of air further needed with each breath or some other measurable quantity. In another example, the LED lights 108 blink or switch on and off in a pattern to indicate progression.

Further examples include that the LED lights 108 change brightness to indicate progression. The brightness level may be used in addition to the number of lights to provide additional communication on progression. For example, the brightness level may indicate the amount of time that the user has remaining, with a lower or higher brightness representing that the time is almost over. A change of LED light 108 color may also be used to communicate time or other information. For example, a LED light 108 may change from green to indicate that time is almost over, and then red to indicate to the user to stop using the device.

In addition to light, or in place of light, other forms of communication may be incorporated. In an example, the spirometer attachment 100 includes a sound device for communicating auditory feedback to a user. The sound may prompt the user to begin breathing or stop breathing. The sound may change or increase in volume as the user uses the device. The sound may include a voice prompt or other type of information during use of the spirometer.

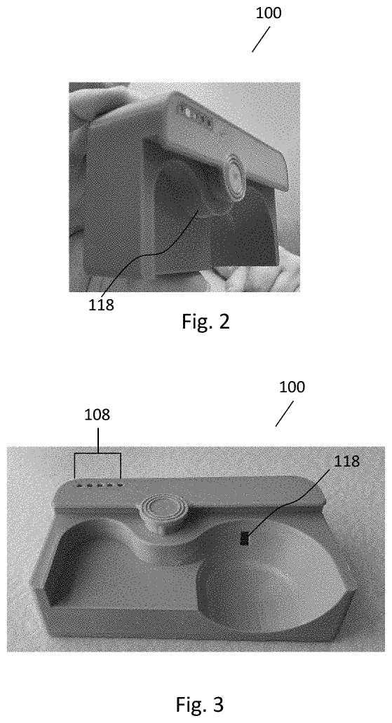

Turning to FIG. 2, a perspective view of the spirometer attachment 100 is shown. The housing 102 includes a recess or cavity in the front. Sidewalls and the top wall curve or undulate to conform to the top and sides of a spirometer.

FIG. 3 also shows the various curves and cutouts within the housing 102 that allow the spirometer attachment 100 to attach to a spirometer. A lip on the front of the housing runs horizontally across the top. The lip slides over the edge of the housing to secure the spirometer attachment 100 to the spirometer. In other examples, securement is achieved with a clip, fastener, screw, hook and loop, tie, strap, or other locking structure.

Also shown in FIG. 3 is a laser range finder 118 that measures breath within the spirometer. In an example, the laser range finder 118 measures a portion of a space within the spirometer. In an example, the portion of space includes the overlapped space of the spirometer that is at least partially surrounded or encased by the housing 102. The overlapped space may be measured by looking at the front of the spirometer or at a pre-determined angle. In other examples, the laser range finder 118 measures other space within the spirometer. Other types of measuring devices may be used. For example, measuring devices may include a light reflecting measuring device or an ultrasound device. As stated previously, the LED light 108 or other lights emit light away from the housing 102 based on the breath data that is measured by the laser range finder 118.

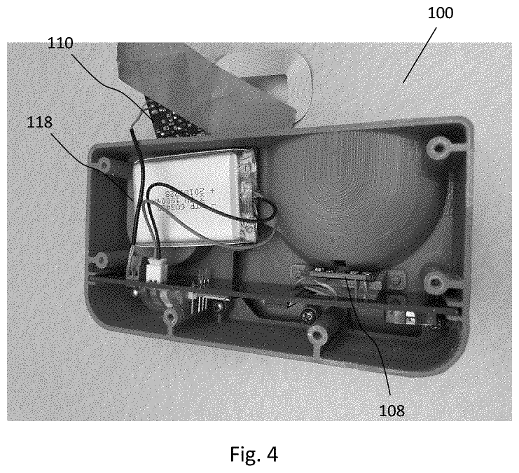

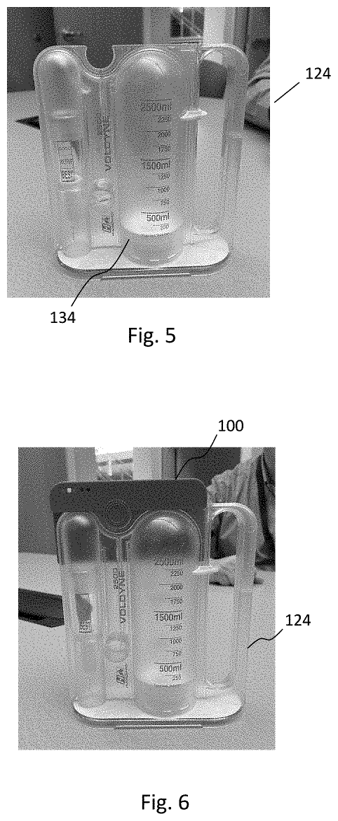

Turning to FIG. 4, a back side of a spirometer attachment 100 is shown. The back side is open to show a partially assembled view of an interior compartment within the housing 102. The housing 102 holds a circuit board 110 and the laser range finder 118. The housing 102 further holds components of the LED lights 108. Additional components may also be held within the housing 102. The circuit board 110 is electronically connected to the LED lights and the laser range finder 118. The circuit board 110 controls the LED lights 108 based on breath data collected by the laser range finder 118. The breath data is used to determine, for example, rate of breath and volume of lung capacity which is then used to determine the type or mode of LED light to be activated.

Further example components in the housing 102 include a battery for powering the at least one LED and the circuit board. In an example, a wireless charger is in the housing 102 and is used to charge the battery. In another example, a wireless transmitter is within the housing 102 and transmits and receives breath related data for determining actuation of the LED lights 108.

An example spirometer is shown in FIG. 100. The spirometer includes a containment. Within the container is a piston 134 that rises and falls as a person breathes into the spirometer, using a mouthpiece (not shown) or other connective device. The spirometer attachment 100 is used to communicate to the person directions that affect the rise and fall of the piston 134.

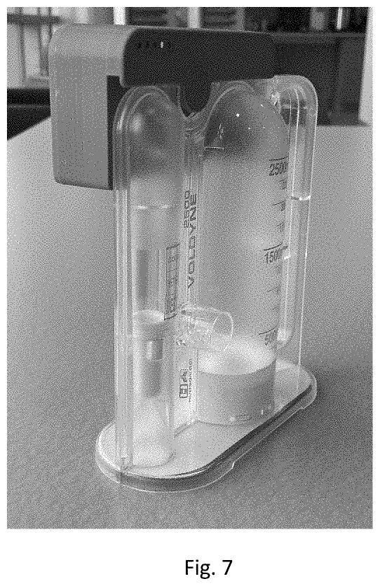

An example setup includes the spirometer attachment 100 attached to the spirometer 124 as shown in FIG. 6. The spirometer 124 includes a mouthpiece (not shown) connected by flexible tubing (not shown) to a containment. An indicator on the spirometer 124 communicates the movement of air into and out of the lungs as measured by the air inhaled through the tubing into the apparatus. The spirometer attachment 100 provides guidance for the user to blow air properly. The spirometer attachment 100 can be completely disengaged from the spirometer 124 for cleaning and storing the device when not in use. Another view of the attachment of the spirometer attachment 100 to the spirometer 124 is shown in FIG. 7. As can be seen, a front side of the spirometer attachment 100 fits over a front side of the spirometer 124.

In another example, the spirometer attachment 100 also provides feedback to hospital staff on the user's progress. The spirometer attachment 100 does this with a communication feature that transmits and exchanges breath related data with an external source.

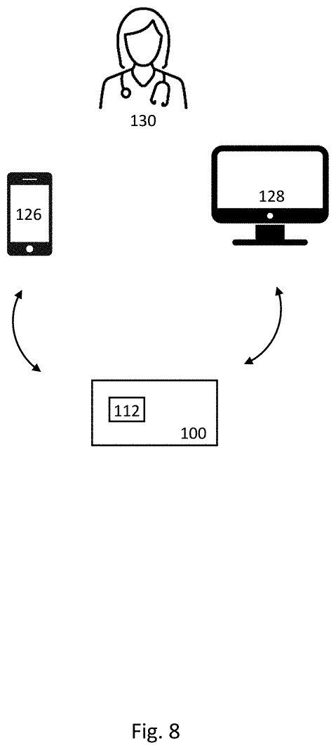

As shown in FIG. 8, the spirometer attachment 100 includes a wireless transmitter 112 that transmits breath related data to a mobile device 126 and a stationary device 128. The mobile device 126 may be a mobile phone, tablet, wearable device (e.g. ear device, wrist device, etc.) or another device that is communicates data related to the spirometer. The stationary device 128 may be a hospital server or other hospital device, stationary computer, cloud, etc. The connection can be any wireless connection, such as Bluetooth, or other known wireless connection that connects to the mobile device 126 and stationary device 128. With this communication, hospital personnel may monitor the data being collected and more readily be available to help a patient.

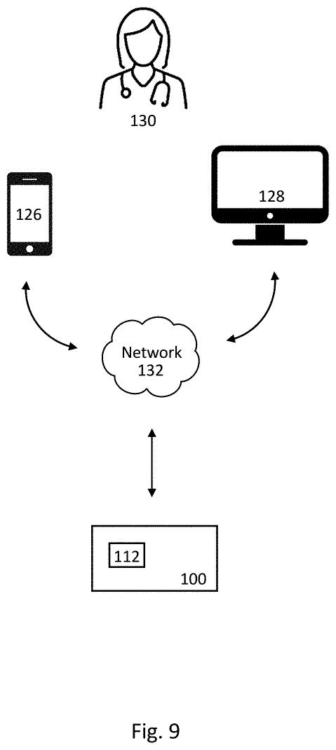

As shown in FIG. 9, the spirometer attachment includes a wireless transmitter 112 that transmits breath related data over a network 132 to a mobile device 126 and a stationary device 128. The network 132 may include a wired or wireless network and may include information that is stored on a server or cloud-based system, or other connection that connects to the mobile device 126 and stationary device 128.

In the description, for purposes of explanation, specific details are set forth in order to provide a thorough understanding of the disclosure. It will be apparent, however, to one skilled in the art that examples consistent with the present disclosure may be practiced without these specific details. Reference in the specification to “an example” or similar language means that a particular feature, structure, or characteristic described in connection with the implementation or example is included in at least that one implementation, but not necessarily in other implementations. The various instances of the phrase “in one implementation” or similar phrases in various places in the specification are not necessarily all referring to the same implementation.

The preceding description has been presented only to illustrate and describe examples of the principles described. This description is not intended to be exhaustive or to limit these principles to any precise form disclosed. Many modifications and variations are possible in light of the above teaching.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A spirometer attachment, comprising:
a housing;
a securement on the housing to removably secure the housing to a spirometer;
a measuring device within the housing to collect breath data from the spirometer;
at least one light-emitting diode (LED) to emit light away from the housing based on the collected breath data; and
a circuit board or controller within the housing to control the light emitted by the LED based on the breath data.

2. The spirometer attachment in claim 1, further comprising a wireless transmitter that transmits and exchanges breath related data with an external source.

3. The spirometer attachment in claim 1, wherein the measuring device includes a light reflecting measuring device, a laser range finder, or an ultrasound device.

4. The spirometer attachment in claim 1, wherein the securement includes a lip, clip, fastener, screw, hook and loop, tie, strap, or locking structure.

5. The spirometer attachment in claim 1, wherein the LED lights up a symbolic shape to users to indicate progress in using the spirometer.

6. The spirometer attachment in claim 1, wherein the housing is water-tight such that it may be removed and sterilized in between uses.

7. The spirometer attachment in claim 1, further comprising a sound device for communicating auditory feedback to a user on use of the spirometer.

8. The spirometer attachment in claim 1, wherein breath related data is transmitted to at least one of a hospital server, mobile device of hospital personnel, or other hospital device.

9. A spirometer attachment, comprising:
a housing;
a securement that removably attaches the housing to a spirometer;
a measuring device within the housing to collect breath data from the spirometer;
at least one LED to emit light away from the housing based on the breath data; and
a circuit board or controller within the housing to control the light emitted by the LED based on the breath data,
wherein the breath data is used to determine at least a breath-related measurement which is then used to determine the type or mode of LED light to be activated.

10. The spirometer attachment of claim 9, wherein the breath-related measurement includes a rate of breath.

11. The spirometer attachment of claim 9, wherein the breath-related measurement includes a volume of lung capacity.

12. The spirometer attachment in claim 9, further comprising:
a battery for powering the at least one LED and the circuit board, and
a wireless charger on the housing that may be charged to charge the battery.

13. The spirometer attachment in claim 9, further comprising a wireless transmitter that transmits and receives breath related data for determining actuation of the at least one LED.

14. The spirometer attachment in claim 9, wherein the housing is watertight.

15. A spirometer attachment, comprising:
a housing;
a securement that removably attaches the housing to a top of the spirometer device with the housing partially inserted within the spirometer;
a laser range finder that measures breath within a portion of the spirometer;
at least one LED to emit light away from the housing based on the breath data; and
a circuit board or controller within the housing to control the light emitted by the LED based on breath data as measured by the laser range finder to guide and teach a user to use the spirometer.
